(19) United States
(12) Patent Application Publication
(10) Pub. No.: US 2017/0354123 A1
(43) Pub. Date: Dec. 14, 2017

(54) System and Method for Real-Time Imaging of Body Composition Traits of Food Animals

(71) Applicants: Doyle E. Wilson, Ames, IA (US); Viren R. Amin, Ames, IA (US)

(72) Inventors: Doyle E. Wilson, Ames, IA (US); Viren R. Amin, Ames, IA (US)

(21) Appl. No.: 15/595,165

(22) Filed: May 15, 2017

Related U.S. Application Data
(60) Provisional application No. 62/349,885, filed on Jun. 14, 2016.

(51) Int. Cl. A01K 29/00; A61B 5/00; A61B 8/08; A61B 8/00; A01K 1/06

(52) U.S. Cl. CPC A01K 29/00; A61B 8/4209; A01K 1/0613; A61B 8/4483; A61B 8/461; A61B 8/08; A61B 5/4872

(57) ABSTRACT

A system and method for scanning a pig (or other food animal) to determine body composition and quality data in real time while the pig (or food animal) is suspended in mid-air by a lift apron in electronic communication with an ultrasound console and computer processor and configured to collect and process “target images” from the pig (or food animal) when a thumb switch activates the processor. The ultrasound probe is vertically displaced from and vertically adjustable relative to the framework so that the ultrasound probe is selectively positioned in relative space. With reference to target images, a processor calculates in real time a quantitative measurement indicative of backfat depth, muscle depth, and intramuscular fat for the pig (or food animal) being scanned.

### REFERENCE TO RELATED APPLICATIONS

This application claims the benefit of provisional patent application U.S. Ser. No. 62/349,885 filed Jun. 14, 2016 titled Scanning and Auto Processing for Fat, Loin, and IMF, and which is incorporated herein in its entirety by reference.

### BACKGROUND OF THE INVENTION

This invention relates generally to animal imaging systems and, more particularly, to an imaging method and system for scanning and displaying key body composition and quality traits of swine (food animals) in real time. Swine or pig is used as an example of a food animal in the remainder of the document as food animals for use with the present invention may also include beef cattle and other livestock.

Genetic swine companies and independent breeders have been scanning live swine for years using real-time ultrasound. Analytic tools have been developed that allow for the accurate and quantitative measurement of three body composition and quality traits from anatomically correct ultrasound images: (1) body composition traits of backfat depth and loin muscle depth, and (2) the quality trait of intramuscular fat. The process can be cumbersome, time consuming, and in some cases, dangerous to both the ultrasound operator and the pig depending on facilities and scanning protocol. A 250-pound pig is best described as a bundle of raw, undirected and explosive energy, constantly fighting and resisting every attempt by the operator to place the probe and capture proper ultrasound images that can later be processed for the body/quality measurement results.

Therefore, it would be desirable to have a method and system for first restricting the movement of the pig before the scanning process by gently lifting the pig off all 4 feet using a lift apron. The lifting method renders the animal virtually immobile. While the pig is suspended on the lift apron, the operator can safely and quickly position the ultrasound transducer in the correct anatomical position to obtain the body composition/quality measure results.

### SUMMARY OF THE INVENTION

A system and method according to the present invention for scanning a pig to determine body composition data in real time while the pig is suspended in mid-air by a lift apron includes an ultrasound probe suspended from a transducer fixture proximate the lift apron and configured to generate a “target image” from the pig while the pig is suspended by operation of the lift apron. A “target image” as referred to in the present disclosure refers to a continuous real-time video stream of successive images showing appropriate tissue interfaces and reference points. The ultrasound probe is vertically displaced from and vertically adjustable relative to the transducer fixture so that the ultrasound probe is selectively positioned, vertically and laterally, in relative space. With reference to the target image, processing algorithms calculate quantitative measurements in real time for backfat depth, muscle depth, and intramuscular fat. An electronic monitor is in data communication with the ultrasound console and configured to display the target image and the quantitative measurements within milliseconds. Body composition/quality measurement results are stored in a computer database for later use.

The present invention adapts the ultrasound image capturing and automatic processing technology from the inventors' previous patents for a pork carcass grading system, called BioQscan® and summarized in the following disclosure. BioQscan® uses an ultrasound scanning system, computer and electronics processing modules, and a transducer fixture for housing the ultrasound probe, a sensor and indicator lights. The operator positions the transducer fixture to “hot” hanging pork carcasses, with carcasses moving at line speeds up to 1,400 per hour. Automatic capturing and processing of sensor data and real-time images happen within millisecond, resulting in quantitative measurements for body composition traits of backfat depth and muscle depth as well as the quality trait of intramuscular fat. These data are stored in a database and can be merged with other packing plant data systems.

Therefore, a general object of this invention is to provide a system and method for scanning a pig to determine body composition and quality measurements data in real time while the pig is suspended and immobilized in mid-air.

Another object of this invention is to provide a system and method for scanning a pig, as aforesaid, having an ultrasound probe mounted in a fixture in the proximity of a pig which is raised in the air by a lift apron. The ultrasound assembly is configured to generate a video stream of internal tissues by scanning through the skin of a pig.

Still another object of this invention is to provide a system and method for scanning a pig, as aforesaid, that determines a body composition including, but not limited to, characteristics such as backfat depth, muscle depth, and intramuscular fat.

A further object of this invention is to provide a system and method for scanning a pig, as aforesaid, that enables a plurality of static images to be stored in a memory for later review.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is an exploded view of a system for scanning and displaying key body composition and quality traits of swine in real time;

FIG. 2 is a front view of the scanning system as in FIG. 1;

FIG. 3 is a side view of the scanning system as in FIG. 1;

FIG. 4a is a front view of the scanning system as in FIG. 1 illustrating the pulley reel assembly and transducer fixture at one lateral position relative to a framework;

FIG. 4b is a front view of the scanning system as in FIG. 1 illustrating the pulley reel assembly and transducer fixture at another lateral position relative to a framework;

FIG. 4c is a front view of the scanning system as in FIG. 1 illustrating the pulley reel assembly and transducer fixture at a vertical position relative to a framework;

FIG. 4d is a front view of the scanning system as in FIG. 1 illustrating the pulley reel assembly and transducer fixture at another vertical position relative to a framework;

FIG. 5 is a perspective view of an animal crate for use with the present invention, illustrated with all gates in closed configurations;

FIG. 6 is a perspective view as in FIG. 5, illustrated with all gates in open configurations;

FIG. 7 is a perspective view of a lift apron for use with the present invention, illustrated in a lowered configuration;

FIG. 8 is a perspective view of the lift apron as in FIG. 7 illustrated in a raised configuration;

FIG. 9 is a front view of the scanning system as in FIG. 1 illustrating the system in use with a pig in the crate.

FIG. 10 is a perspective view of an ultrasound transducer fixture according to the present invention;

FIG. 11a is a front view of the transducer fixture as in FIG. 10;

FIG. 11b is a front view of the monitor displaying an ultrasound image in use;

FIG. 12 is a flowchart illustrating the method according to the present invention;

FIG. 13a is a flowchart illustrating the method according to the present invention;

FIG. 13b is a flowchart illustrating the method according to the present invention; and

FIG. 14 is a block diagram of the electronic components of the present invention.

### DESCRIPTION OF THE PREFERRED EMBODIMENT

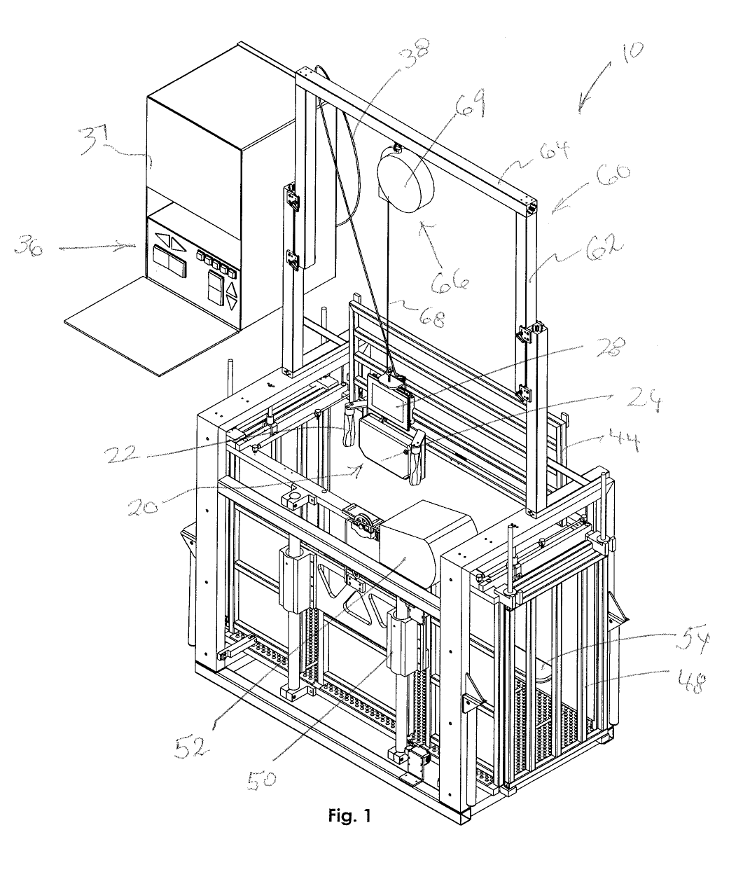

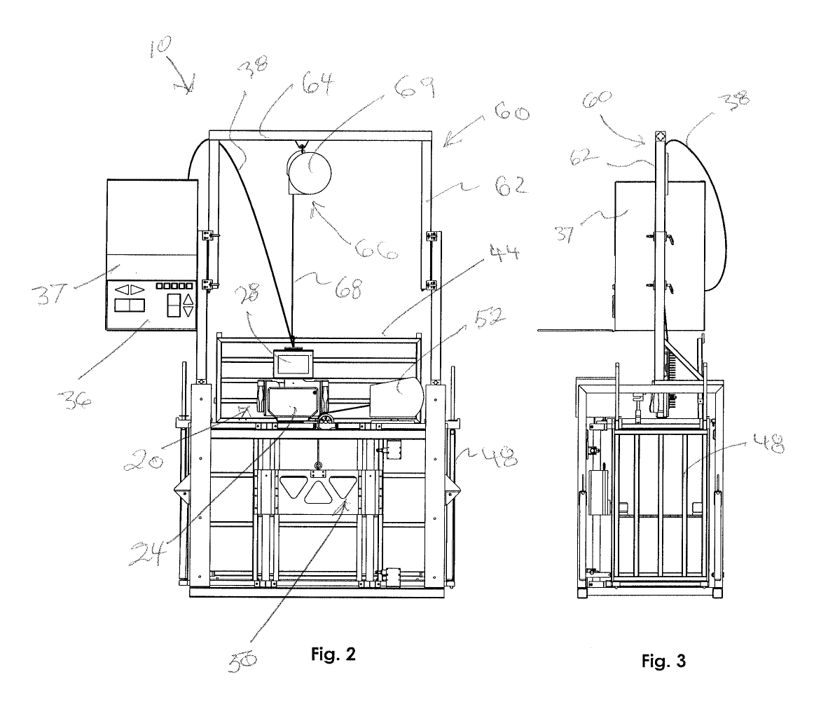

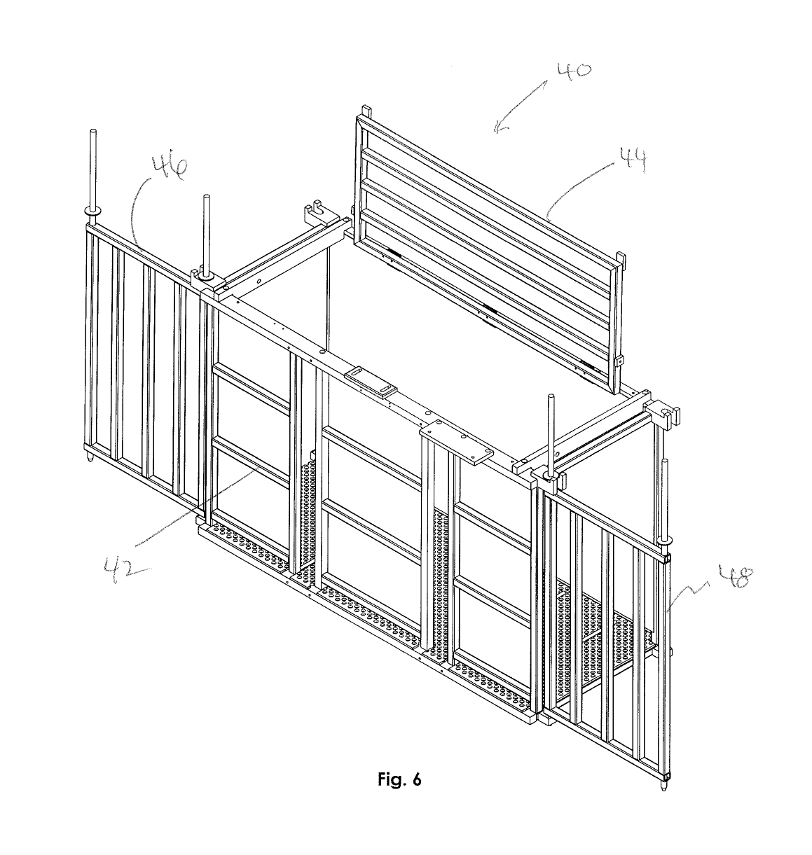

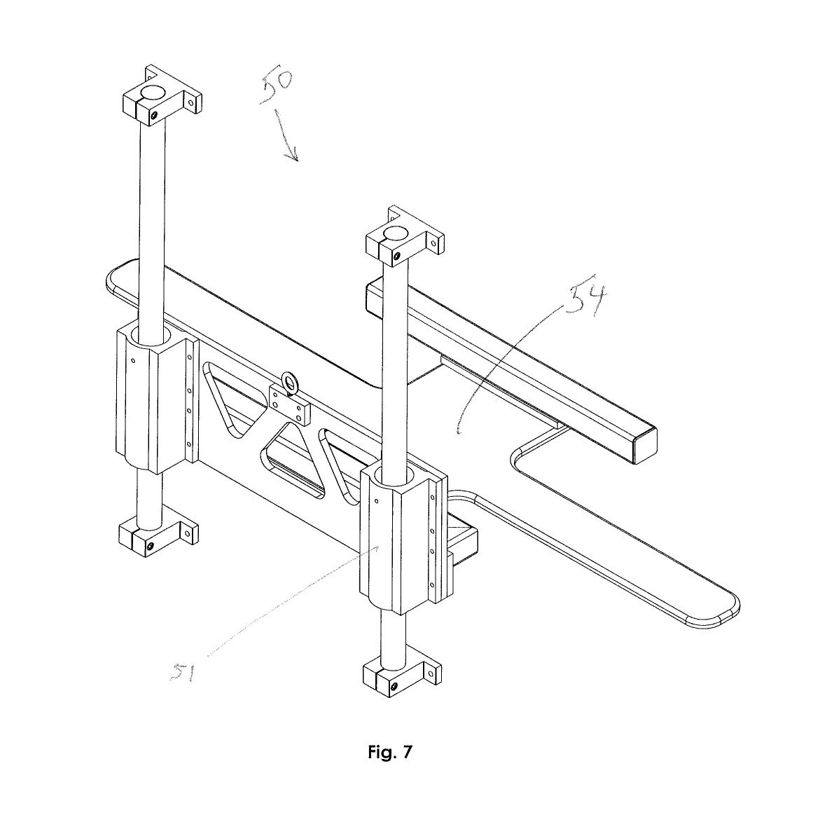

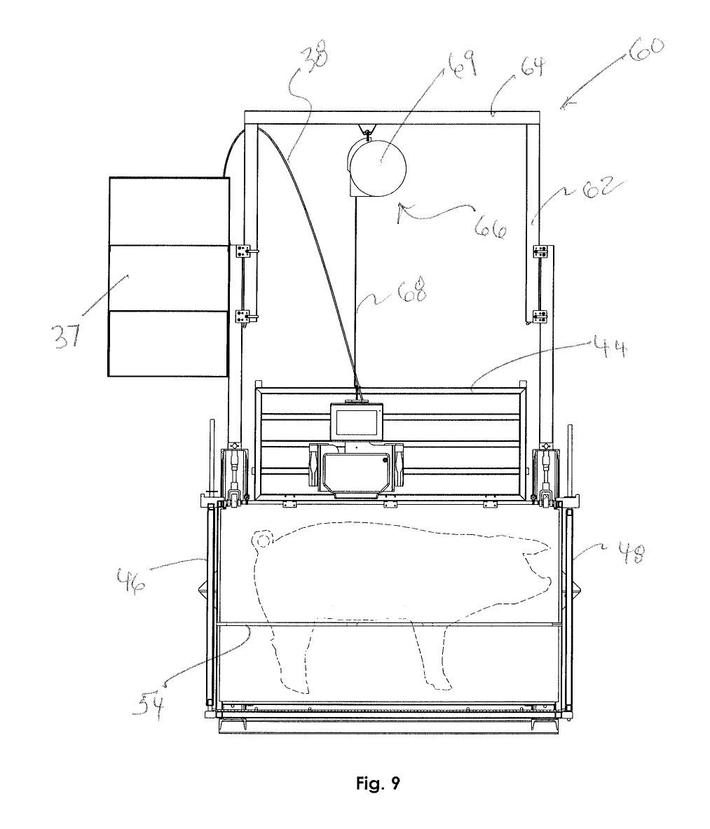

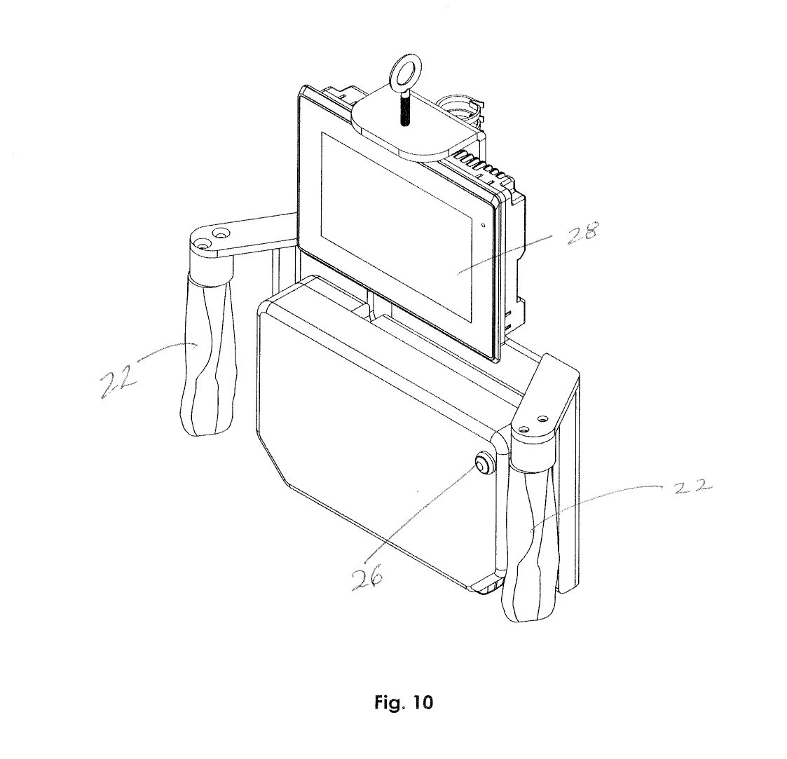

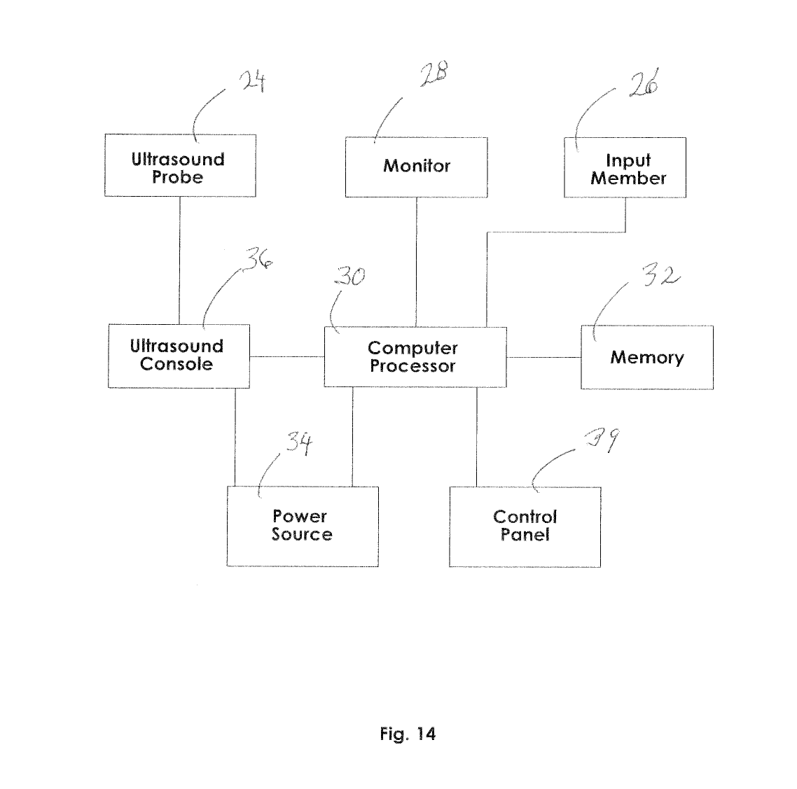

A system and method for imaging swine according to a preferred embodiment of the present invention will now be described in detail with reference to FIGS. 1 to 14 of the accompanying drawings. The imaging system 10 includes an ultrasound probe 24 mounted to a transducer fixture 20 and an electronic monitor 28 in data communication with the computer processor 30. The imaging system 10 also includes an ultrasound console 36 (also referred to merely as “the console”), which preferably includes a computer processor 30 configured to determine backfat depth, muscle depth, and intramuscular fat of a pig while the pig is suspended and immobilized above the ground by a lift apron using data from a video stream generated by the ultrasound probe 24. In other words, the ultrasound probe 24 is in data communication with the console 36 and computer processor 30 via a data cable 38 or bundle or cords, the console 36 being configured to generate real-time images from data received from the probe 24. The console 36 may be situated in an electronics cabinet attached to side brace 62 of the main framework 60 (FIG. 1) or upon another table or shelf adjacent the framework 60.

In an exemplary embodiment, the imaging system 10 of the present invention is intended for use with a livestock crate 40 for restricting movement of a pig to be scanned and a lift apron 50 positioned inside the crate 40 for selectively lifting the pig into the air prior to being scanned. The crate 40 may include a plurality of fence-like brace members 42 arranged to form a box or individual stock pen, each brace member 42 being spaced apart from one another so as to leave ample openings for ventilation while still inhibiting the pig from escaping. The crate 40 may include a top gate 44 pivotally movable between an open configuration enabling access to the interior of the crate 40 and a closed configuration not allowing access to the crate interior. In use, the top gate 44 is opened after the pig to be scanned is lifted upwardly in the crate 40 by the lift apron 50. The crate 40 may also include pivotal front 46 and rear 48 gates configured to allow a pig to enter and exit the crate 40, respectively. Alternatively, one or both end gates may be configured to move vertically in a guillotine fashion.

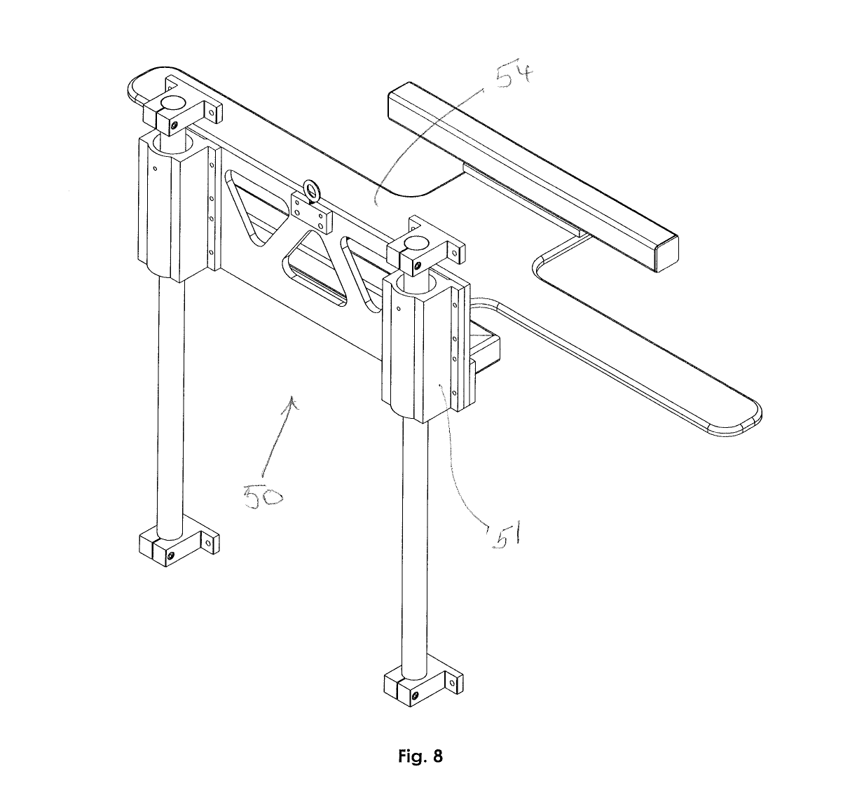

The lift apron 50 is a device similar to a jack that may be situated within the interior of the crate 40 and configured to raise or lift a pig upwardly and be suspended above the ground when energized. More particularly, the lift apron 50 may include an elevator member 51 that is selectively movable between raised and lowered configurations and includes a lift plate 54 coupled to the elevator member 51. The lift plate 54 may have an elongate and planar configuration that is adapted to engage the underbelly of a pig that is standing above the lowered lift plate 54. The lift apron 50 may be operated by a lift motor 52 (FIG. 1) and is configured to raise the pig off the ground when energized (see FIGS. 7 to 9). It is understood that the elevator member 51 may be taken from a group consisting of a hydraulic lift, pneumatic lift, motor driven lift, and an electric lift.

The ultrasound probe 24 is an electronic device configured to generate a video streaming series of images of the internal body structures of a human person or animal. More particularly, ultrasound is an imaging technique that uses ultrasonic pulses (sound waves) to generate electronic images of internal structures such as muscles, tendons, organs, and the like. In use, the ultrasound probe 24 requires a couplant to allow transmission of the sound waves from the ultrasound probe 24 through the skin of a human person or animal to the internal body structures below the skin. The couplant material used to allow transmission of the sound waves may be water, vegetable oil or an appropriate acoustical gel. Images may be generated in the form of a real-time video stream when the ultrasound probe 24 is positioned on the skin of a body proximate the tissues or organs to be scanned.

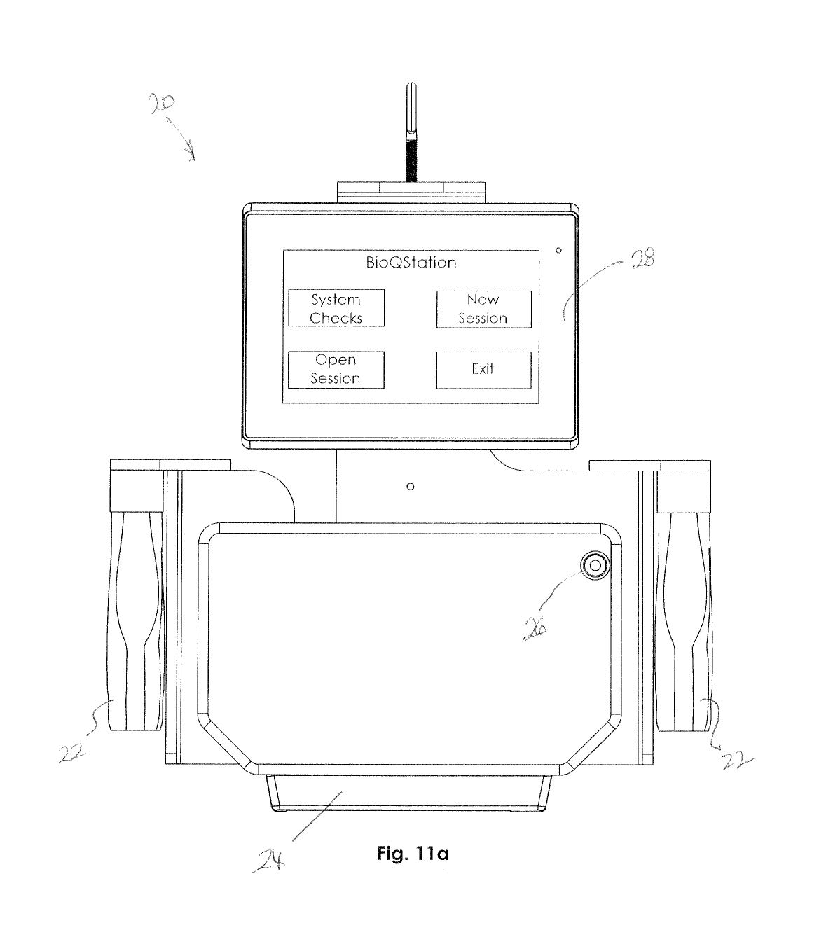

In the present invention, the ultrasound probe 24 may include an input member 26 (FIG. 10) configured, when actuated, to begin capturing a subsequent series of images. The input member 26 may be a momentary thumb switch on the ultrasound probe 24 itself, on the transducer fixture 20, or on a keyboard associated with the ultrasound console 36 electrically connected to or in wireless communication with the computer processor 30, ultrasound probe 24, or an on-screen touch button on a touch screen monitor 28. The ultrasound console 36 and processor 30 are configured to receive and process the streaming series of images being collected and generated by the ultrasound probe 24.

The ultrasound probe 24 is electrically connected to and in data communication with ultrasound console 36, computer processor 30 and a video monitor 28 which may also be referred to as a video display or computer display. The video monitor 28 may be integrally connected to an upper edge of the ultrasound probe 24 and, preferably, integrated therewith as an ultrasound transducer fixture 20, or referred to merely as a “transducer fixture.” It is understood that the video monitor 28 and ultrasound probe 24 of the transducer fixture 20 are in data communication with the ultrasound console 36 and under its control via operation of a computer processor 30. An integrated transducer fixture 20 enables a user to view instructions, option menus, and images being generated (FIG. 10). The integrated transducer fixture 20 may be suspended in the air and configured to be manipulated spatially by a user so as to be placed in contact with a pig suspended in the air to be scanned as will be described below. The transducer fixture 20 may include one or more handles 22 configured to enable a user to grasp and move the transducer fixture 20 vertically and horizontally. As introduced previously, the ultrasound transducer fixture 20 may also include a processor 30 and a non-volatile memory 32 in data communication with the processor 30, the memory 32 being configured to store data and a plurality of programming instructions to be executed by the processor 30 as will be described later in more detail. The processor 30 and related electronics may be powered by a power source 34 such as a battery or by AC power via a power cord.

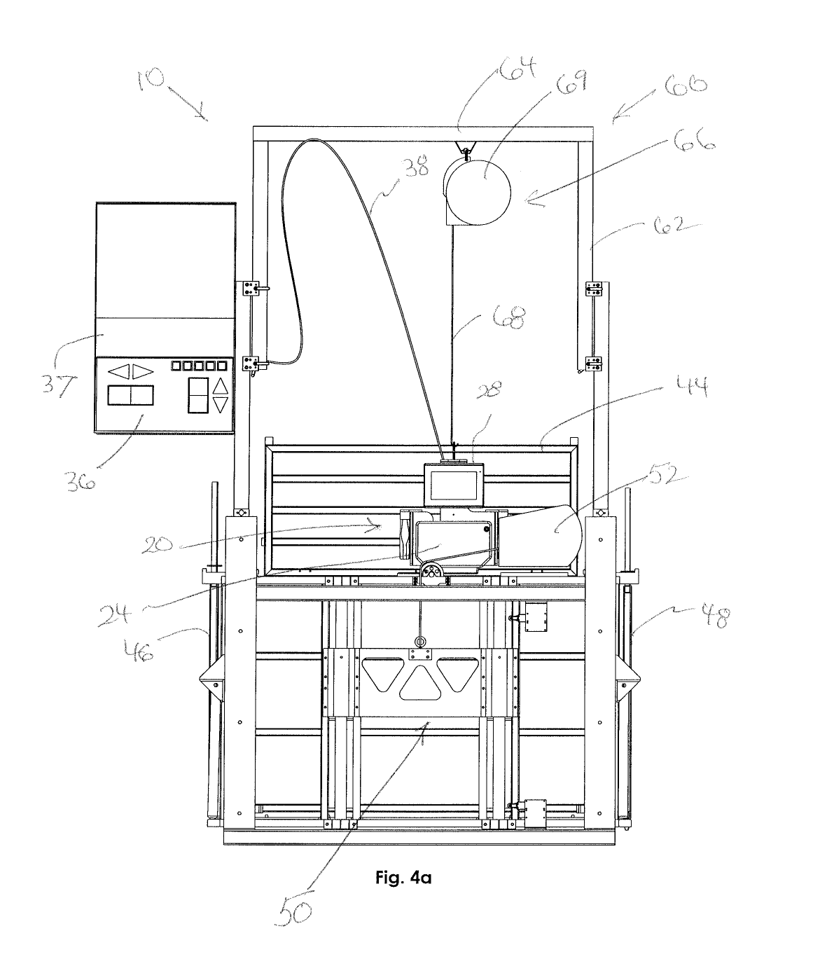

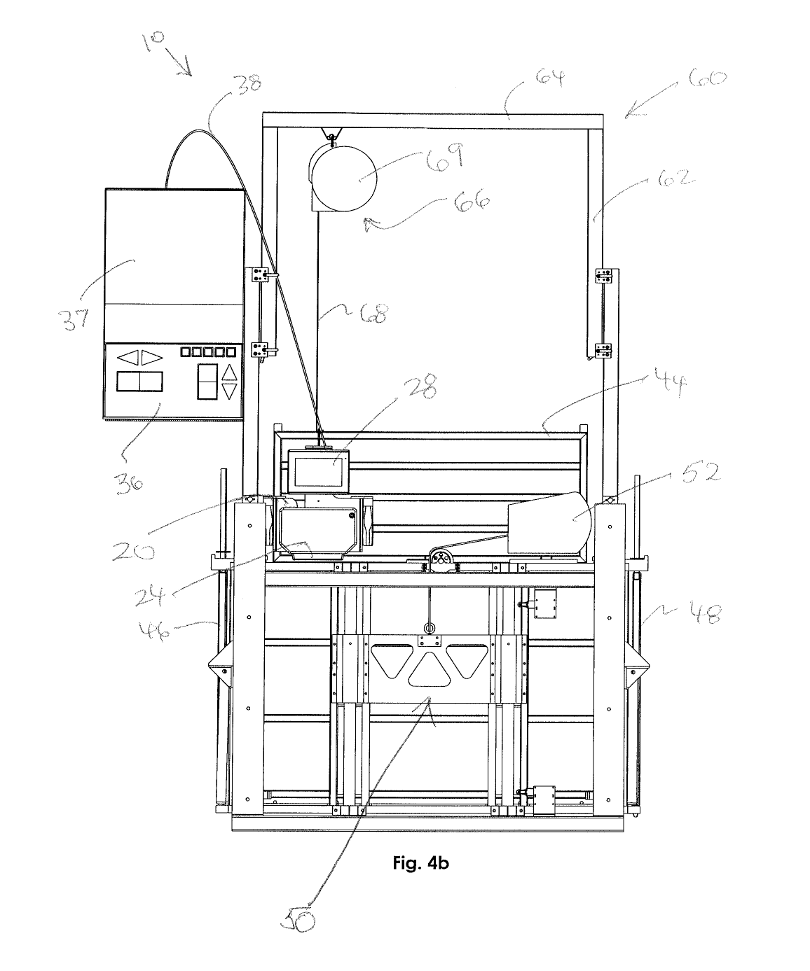

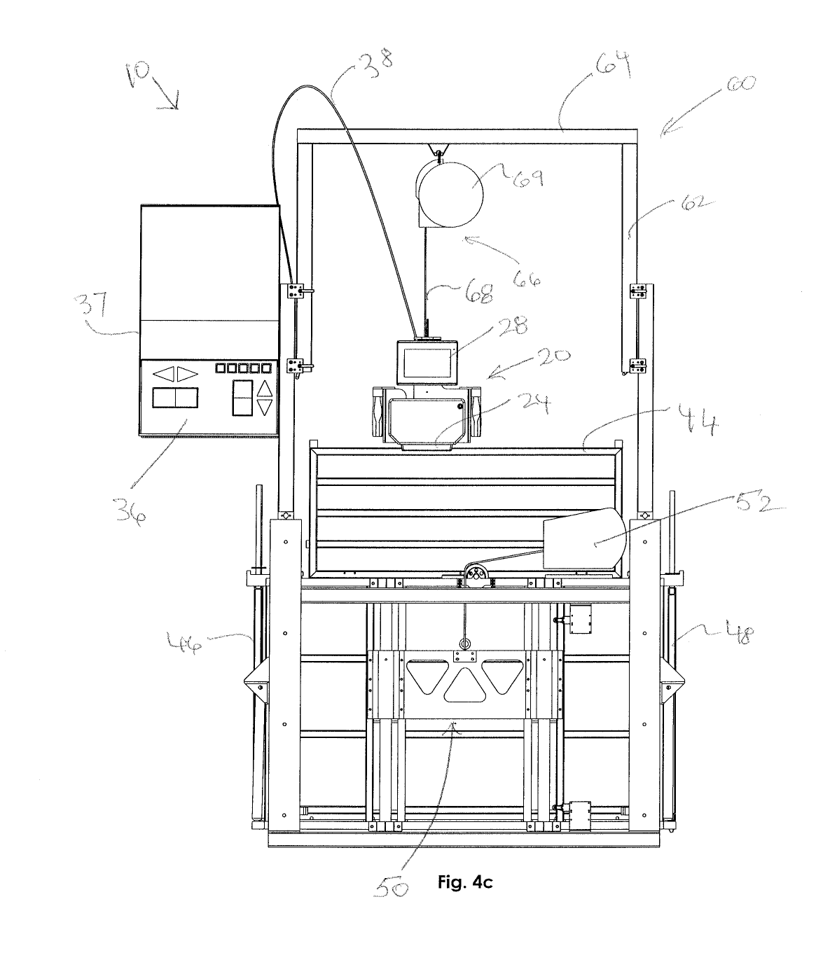

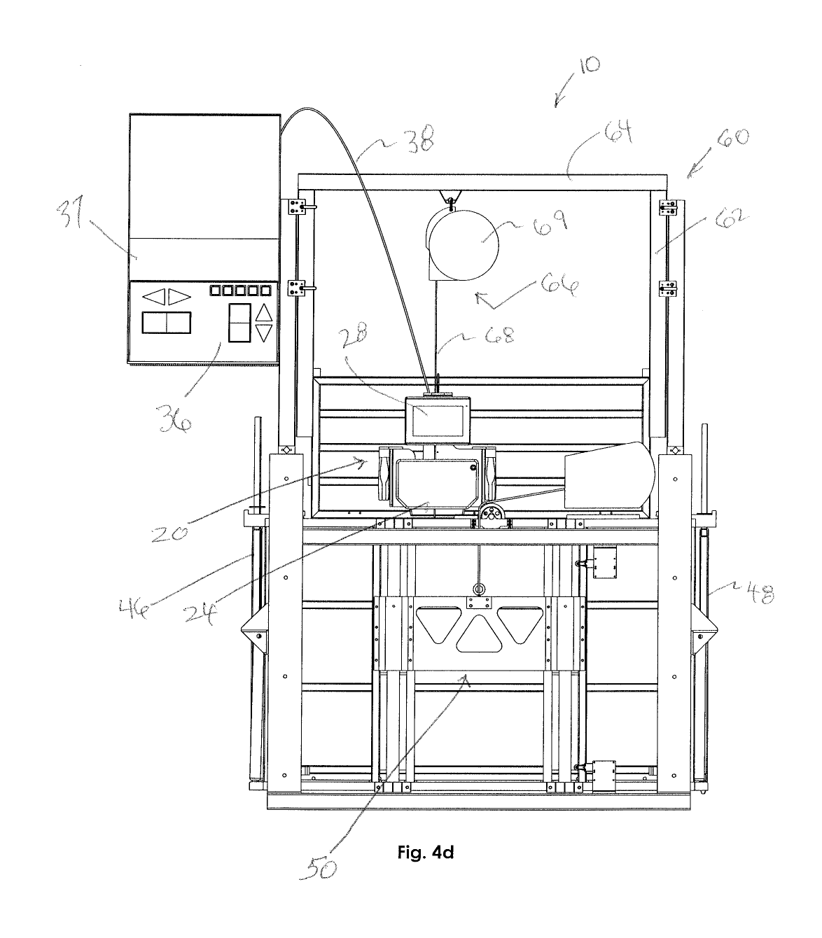

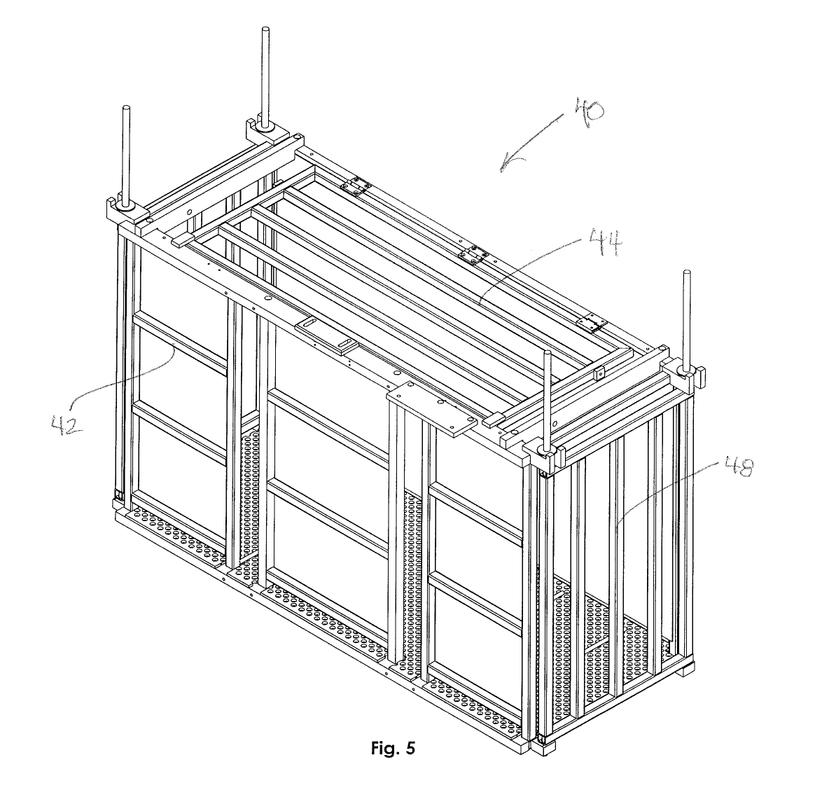

Preferably, the ultrasound transducer fixture 20 is suspended from a framework 60 and configured to move both vertically and laterally. More particularly, the framework 60 may include one or more upstanding side braces 62 and an upper brace 64 extending between free ends of the side braces 62. In an embodiment, the side braces 62 may be length adjustable such that the framework 60 is height adjustable (compare FIGS. 4c and 4d). A bottom surface of the upper brace 64 may define a channel extending between the side braces 62 of the framework 60.

The ultrasound transducer fixture 20 is configured to move vertically and laterally relative to the upper brace 64. More particularly, a pulley reel assembly 66 may be operatively coupled to the channel and, therefore, to the framework 60. It is understood that a chain drive, belt drive, gear mechanism, or the like (not shown) may be positioned in the channel that is operatively coupled to the pulley reel assembly 66 and configured to move laterally therealong when energized. The pulley reel assembly 66 may include a cable 68 that is selectively movable between a stowed configuration inside a pulley reel housing 69 and a deployed configuration substantially outside the pulley reel housing 69. A distal end of the cable 68 may be coupled to the ultrasound transducer fixture 20. The pulley reel assembly 66 may be configured as a counterbalance that enables the transducer fixture 20 to be suspended in the ambient air. In other words, the transducer fixture 20 may be pulled downwardly or pushed upwardly by the user and then it holds its position in space—becoming virtually weightless.

In an embodiment, the pulley reel assembly 66 may be moved manually by a user in a lateral position along the channel or moved via the cable 68 between stowed and deployed configurations. Alternatively, an electric or electronic device (not shown) could be added to the controls 39 allowing electronic movements laterally and stowed or deployed configurations of the pulley reel assembly 66. In an embodiment, the console 36 may be displaced from the transducer fixture 20, such as mounted or positioned on an adjacent cabinet. Further, the console 36 may be electrically connected to the transducer fixture 20 via one or more data cables 38 configured to transfer data from the transducer fixture 20 through the console 36 to the computer processor 30 for long term storage.

The ultrasound probe 24, when connected to the ultrasound console 36 and powered up, generates a continuous real-time video stream of images, and when not in contact with an object, the video stream shows blank images. Under software control, the streaming images (that is, real-time video) may be displayed on the screen of the monitor 28 which is attached to the transducer fixture 20.

With a couplant applied to the skin and when the transducer fixture 20 is moved into a desired position touching the skin of the pig, the video stream shows internal tissues of that portion of the pig anatomy being scanned by the ultrasound probe 24. When the user views a “target image” showing the desired tissue interfaces and anatomical reference points, the input member 26 may be pressed to enable the computer processor 30. When enabled, the computer processor 30 captures the series of real-time images where they may be mathematically processed, displayed, archived, or stored for later review.

Specifically, the images may be stored in a non-volatile memory 32 for later review, archival, or printing. From the captured image stream data, the computer processor 30 is programmed to determine body composition and quality traits, such as backfat depth, muscle depth, and intramuscular fat.

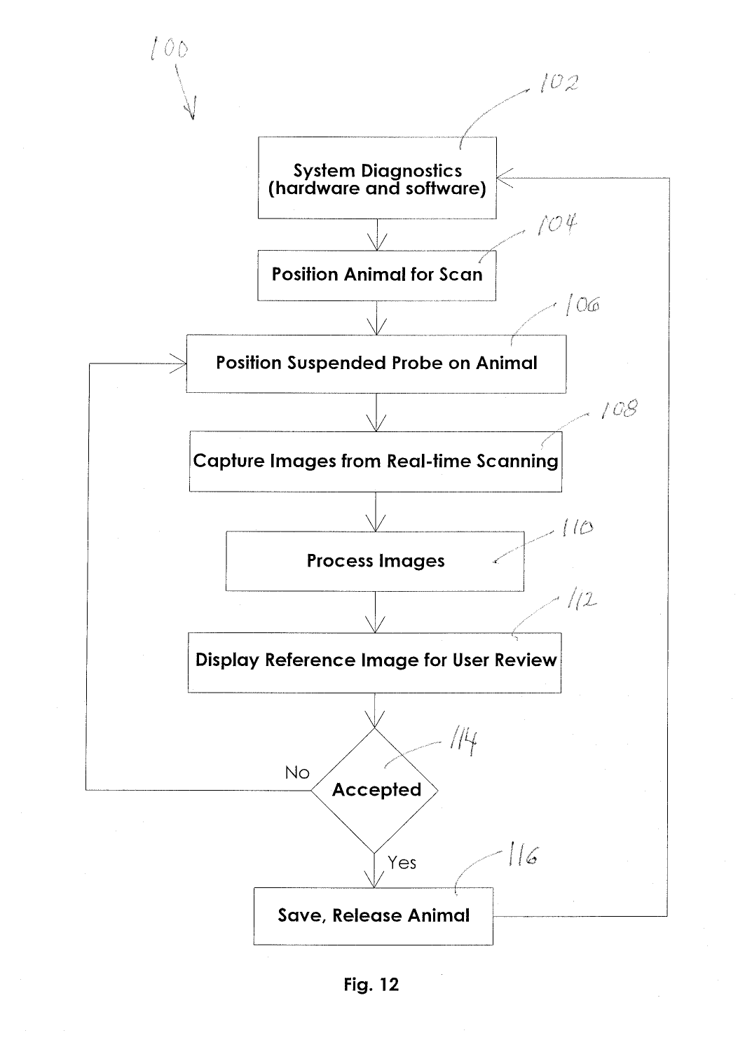

A process 100 illustrating the steps of the method for scanning a pig to determine body composition data according to the present invention is shown in FIG. 12. At step 102, the processor 30 performs a series of diagnostic tests to verify operation of the ultrasound console 36 communication, input member 26, and other components. Then, at step 104, the pig to be scanned is properly positioned in the crate 40 with top gate 44 closed before raising the pig on the lift apron. After the pig is raised, the top gate 44 can be opened to display the back of the pig. At step 106, a user moves the transducer fixture laterally and vertically relative to the framework 60 and positions the ultrasound probe 24 on the skin of the pig. A video stream showing the targeted section of the animal is displayed on the video monitor 28 when the ultrasound probe 24 is energized. At step 108, when the user presses input member 26 indicating that a target image is achieved on the display, images from the video steam are collected by the computer processor 30 under software control.

Then, the processor 30, under program control, is configured to process the captured images and determine body composition traits as described above and to display that information on the video monitor 28 for real-time review by the operator. At step 114, the processor 30 determines if the operator has accepted the images and, if so, saves the data to memory at step 116 and the user lowers the pig for release. If not accepted, however, the process returns to step 106 to again collect images.

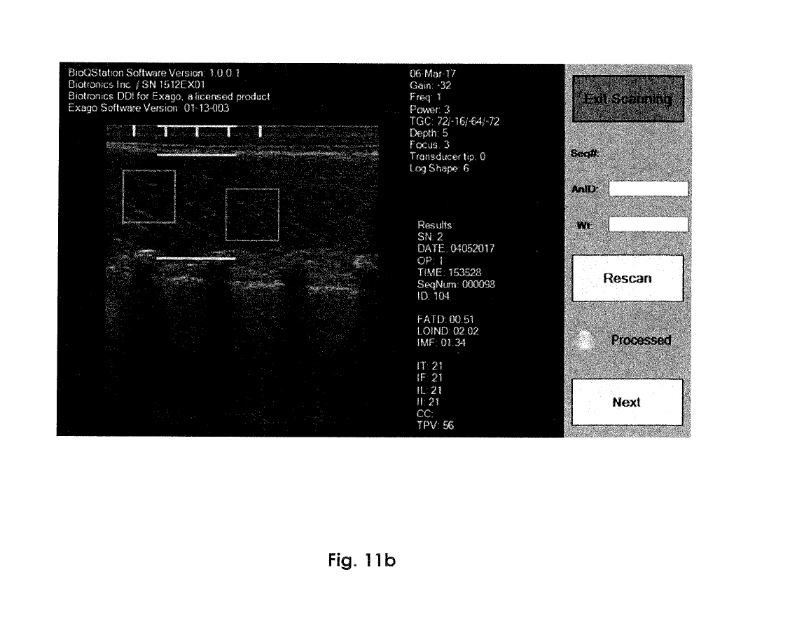

With specific reference to the operation of the software and methodology concerning review and approval of captured image data (step 114), it is noted that after capturing and processing a predetermined number of images for composition and quality measurements, a single representative reference image appears on the monitor 28. A series of overlays displayed on the reference image shows the operator exactly where and how the measurements were made for the current pig that was just scanned. At this point, the operator makes an assessment as to whether the scanning and processing yield a correct interpretation in regards to the body composition and quality measures. If correct, the operator touches a “Next” button on the touch screen display 28 to prepare for the next pig. Otherwise, he presses a “Rescan” button and program control returns to step 106 (FIG. 11b).

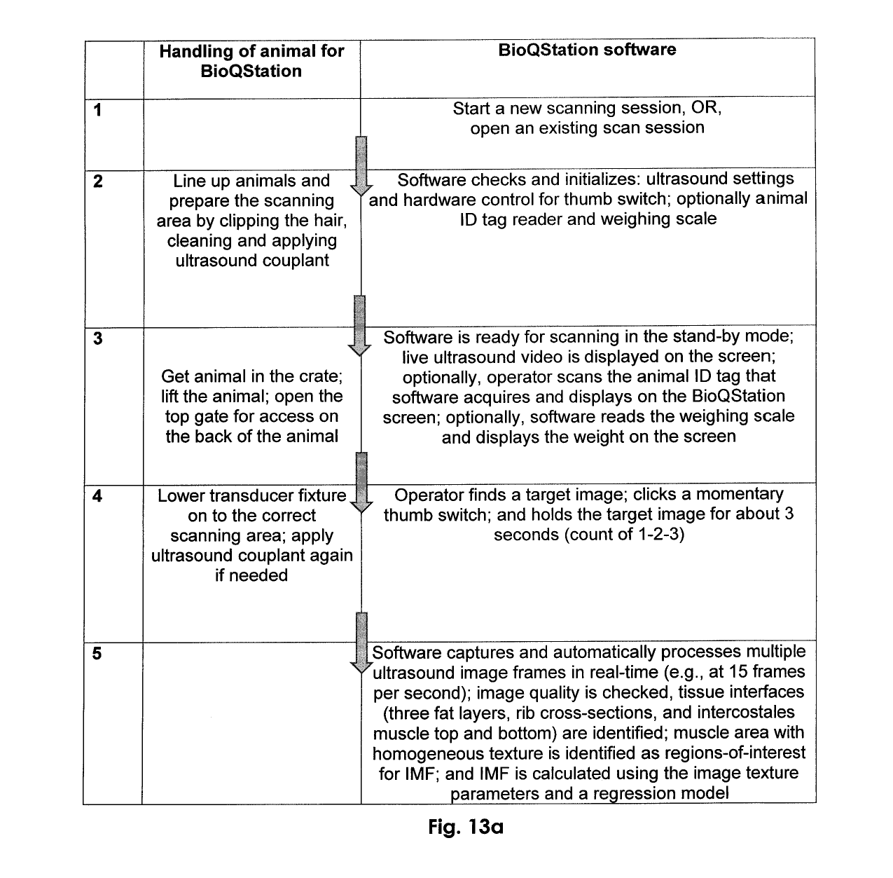

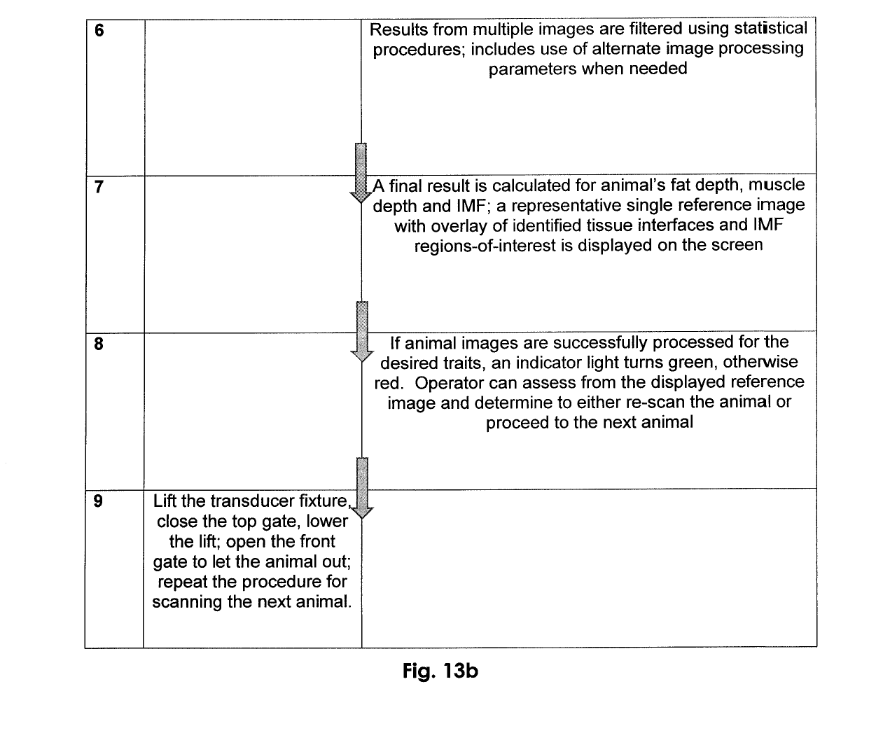

FIGS. 13a and 13b expound upon each step described above and this disclosure is incorporated into the present specification in its entirety.

It is understood that while certain forms of this invention have been illustrated and described, it is not limited thereto except insofar as such limitations are included in the following claims and allowable functional equivalents thereof.

1. A system for scanning a food animal to determine body composition and quality data in real time while the food animal is raised and mounted in a casing called a transducer fixture, suspended in mid-air by a lift apron, comprising:
an ultrasound probe suspended in ambient air from a framework proximate the lift apron and configured to generate a “target image” from the pig while the pig is suspended by operation of the lift apron;
wherein said ultrasound probe is vertically displaced from and vertically adjustable relative to said framework so that said ultrasound probe is selectively positioned;
a computer processor configured to determine, from said target image, at least one quantitative measurement indicative of a body composition of the pig in real time;
a monitor in data communication with said processor and configured to display said target image and said at least one quantitative measurement.

2. The system as in claim 1, wherein:
said target image includes a video stream generated in real time by said ultrasound probe and ultrasound console;
said transducer fixture includes an input member configured to capture image data from said video stream;
said computer processor is configured to determine, from said captured image data, said at least one quantitative measurement indicative of a body composition of the pig in real time;
said monitor is in data communication with said processor and configured to display said captured image data and said at least one quantitative measurement.

3. The system as in claim 2, wherein:
said ultrasound probe and said monitor are integrated and positioned in an ultrasound transducer fixture;
said ultrasound console and computer processor are electrically connected to communicate and stream images under software control;
a non-volatile memory electrically connected to said processor and configured to store a plurality of static images associated with said target image.

4. The system as in claim 3, wherein said input member is a thumb switch positioned on said ultrasound transducer fixture or a touch button on the monitor (touch screen).

5. The system as in claim 2, further comprising:
a non-volatile memory in data communication with said processor and configured to store programming instructions for execution by said processor;
programming instructions that, when executed by said processor, cause said processor to capture image data from said video stream of said target image;
programming instructions that, when executed by said processor, cause said processor to calculate, using said captured image data, said at least one quantitative measurement indicative of a body composition of the pig.

6. The system as in claim 5, wherein said at least one quantitative measurement is taken from a group including traits of backfat depth, muscle depth, and intramuscular fat.

7. The system as in claim 1, wherein said at least one quantitative measurement is taken from a group including traits of backfat depth, muscle depth, and intramuscular fat.

8. The system as in claim 1, wherein said transducer fixture is laterally movable along a channel defined by upper brace of said framework.

9. The system as in claim 8, wherein said framework includes:
a pulley reel assembly coupled to said upper brace and movable laterally along said channel, said pulley reel assembly including a cable selectively movable between a stowed configuration inside a pulley housing and a deployed configuration extending away from said pulley housing;
wherein said ultrasound transducer fixture is coupled to a distal end of said cable such that said ultrasound transducer fixture is laterally and vertically movable relative to said upper brace.

10. The system as in claim 1, further comprising a control assembly operatively coupled to said framework and including electronic controls electrically connected to said pulley reel assembly with said transducer fixture attached, said electronic controls configured to cause said pulley reel assembly to selectively move laterally along said channel defined by upper brace of said framework and to actuate said cable between said stowed and deployed configurations.

11. A method for scanning a food animal to determine body composition data in real time while the food animal is suspended in mid-air by a lift apron, comprising:
suspending an ultrasound probe from a framework proximate the lift apron and configured to generate a “target image” from the pig while the pig is suspended in air by operation of the lift apron;
wherein said ultrasound probe is vertically displaced from and vertically adjustable relative to said framework so that said ultrasound probe is selectively positioned;
determining from said target image at least one quantitative measurement indicative of a body composition of the pig in real time;
displaying said target image and said at least one quantitative measurement on a monitor.

12. The method as in claim 11, wherein:
said target image includes a video stream generated in real time by said ultrasound probe;
said method further comprising:
capturing image data in real time from said video stream communicating image data to said ultrasound console and said computer processor;
determining in real time said at least one quantitative measurement from said captured image data that is indicative of a body composition of the pig;
displaying said captured image data and said at least one quantitative measurement on an electronic monitor.

13. The method as in claim 12, wherein:
said ultrasound probe and said monitor are integrated and positioned in an ultrasound transducer fixture;
said method further comprising storing a plurality captured image data associated with said target image in a non-volatile memory.

14. The method as in claim 12, wherein said ultrasound transducer fixture includes a thumb switch positioned on said ultrasound transducer fixture that is configured to actuate said capturing image data from said video stream.

15. The method as in claim 11, wherein said at least one quantitative measurement is taken from a group including traits of backfat depth, muscle depth, and intramuscular fat.

16. The method as in claim 11, wherein said ultrasound probe is laterally movable along a channel defined along an upper brace of said framework.

17. The method as in claim 16, wherein said framework includes:
a pulley reel assembly coupled to said transducer fixture and laterally movable along said channel, said pulley reel assembly including a cable selectively movable between a stowed configuration inside a pulley housing and a deployed configuration extending away from said pulley housing;
wherein said ultrasound probe is coupled to a distal end of said cable such that said ultrasound probe is laterally and vertically movable relative to said framework.

18. The imaging system as in claim 1, further comprising:
a lift apron having an elevator member selectively movable between lowered and raised configurations, said lift apron having a lift plate coupled to said elevator member;
wherein said lift plate has an elongate planar configuration for engaging a ventral side of the pig, whereby the pig is lifted upwardly when the elevator is energized and moved to the raised configuration.

19. The imaging system as in claim 18, wherein said elevator member is one of a hydraulic lift, a pneumatic lift, a motorized lift, or an electric lift.

20. The imaging system as in claim 18, further comprising a stock crate having a plurality of brace members configured to restrain movement of a pig therein, said stock crate having a front gate, rear gate, and top gate each selectively movable between open and closed configurations;
wherein said lift apron is positioned inside said stock crate.
